(19) United States
(12) Patent Application Publication
(10) Pub. No.: US 2025/0221846 A1
(43) Pub. Date: Jul. 10, 2025

(54) FLUID COLLECTION ASSEMBLIES INCLUDING A FLUID IMPEREABLE BARRIER DEFINING AT LEAST ONE OF A FLATTENED SUMP OR AT LEAST ONE APERTURE

(71) Applicant: PUREWICK CORPORATION, Covington, GA (US)

(72) Inventors: Jonathan Robichaud, Decatur, GA (US); Vinayaka Kulkarni, Bangalore (IN); Matthew Jordan Rothberg, Atlanta, GA (US); Seth Schneider, Social Circle, GA (US)

(21) Appl. No.: 18/851,197

(22) PCT Filed: Mar. 28, 2022
(86) PCT No.: PCT/US2022/022111

§ 371 Date: Sep. 26, 2024

(51) Int. Cl. A61F 5/455

(52) U.S. Cl. CPC A61F 5/455

(57) ABSTRACT

An example fluid collection assembly includes a fluid impermeable barrier and at least one porous material disposed in the fluid impermeable barrier. The fluid impermeable barrier includes a proximal end region and a distal end region opposite the proximal end region. The fluid impermeable barrier defines a chamber, an opening, and a fluid outlet. The fluid impermeable barrier also defines at least one of a flattened sump at or near the distal end region or at least one aperture. The at least one aperture formed in the fluid impermeable barrier may form at least one of at least one first coupling configured to be attached to at least one second coupling attached to the porous material, at least one vent at or near the proximal end region, or at least one indentation formed in at least one lateral flange.

### BACKGROUND

A person or animal may have limited or impaired mobility so typical urination processes are challenging or impossible. For example, a person may experience or have a disability that impairs mobility. A person may have restricted travel conditions such as those experienced by pilots, drivers, and workers in hazardous areas. Additionally, sometimes bodily fluids collection is needed for monitoring purposes or clinical testing.

Urinary catheters, such as a Foley catheter, can address some of these circumstances, such as incontinence. Unfortunately, urinary catheters can be uncomfortable, painful, and can lead to complications, such as infections. Additionally, bed pans, which are receptacles used for the toileting of bedridden patients are sometimes used. However, bedpans can be prone to discomfort, spills, and other hygiene issues.

### SUMMARY

Embodiments are directed to fluid collection assemblies having fluid impermeable barriers defining at least one of a flattened sump or at least one aperture, fluid collection systems including the same, and methods of using and forming the same. In an embodiment, a fluid collection assembly is disclosed. The fluid collection assembly includes a fluid impermeable barrier having a proximal end region and a distal end region opposite the proximal end region. The fluid impermeable barrier also at least defines a chamber, at least one opening, and a fluid outlet at or near the distal end region. The fluid impermeable barrier defining at least one flatten sump at the distal end region thereof or at least one aperture. The fluid collection assembly also includes at least one porous material disposed in the chamber. When the fluid impermeable barrier includes the at least one aperture, the at least one aperture forms at least one of at least one first coupling configured to be attached to at least one second coupling that is attached to the at least one porous material, at least one vent at or near the proximal end region, or at least one indentation formed in at least one lateral flange extending laterally outwardly from portions of the fluid impermeable barrier defining the chamber.

In an embodiment, a fluid collection system is disclosed. The fluid collection system includes a fluid collection assembly. The fluid collection assembly includes a fluid impermeable barrier having a proximal end region and a distal end region opposite the proximal end region. The fluid impermeable barrier also at least defines a chamber, at least one opening, and a fluid outlet at or near the distal end region. The fluid impermeable barrier defining at least one flatten sump at the distal end region thereof or at least one aperture. The fluid collection assembly also includes at least one porous material disposed in the chamber. When the fluid impermeable barrier includes the at least one aperture, the at least one aperture includes at least one of at least one first coupling configured to be attached to at least one second coupling that is attached to the at least one porous material, at least one vent at or near the proximal end region, or at least one indentation formed in at least one lateral flange extending laterally outwardly from portions of the fluid impermeable barrier defining the chamber. The fluid collection system also includes a fluid storage container and a vacuum source. The chamber of the fluid collection assembly, the fluid storage container, and the vacuum source are in fluid communication with each that, when one or more bodily fluids are present in the chamber, a suction provided from the vacuum source to the chamber of the fluid collection assembly removes the one or more bodily fluids from the chamber and deposits the bodily fluids in the fluid storage container.

Features from any of the disclosed embodiments may be used in combination with one another, without limitation. In addition, other features and advantages of the present disclosure will become apparent to those of ordinary skill in the art through consideration of the following detailed description and the accompanying drawings.

### BRIEF DESCRIPTION OF THE DRAWINGS

The drawings illustrate several embodiments of the present disclosure, wherein identical reference numerals refer to identical or similar elements or features in different views or embodiments shown in the drawings.

FIG. 1A is a side elevational view of a fluid collection assembly, according to an embodiment.

FIG. 1B is a cross-sectional schematic of the fluid collection assembly taken along plane IB-IB, according to an embodiment.

FIG. 2 is a cross-sectional schematic of a fluid collection assembly, according to an embodiment.

FIGS. 3A and 3B are cross-sectional schematics illustrating a method of securing a porous material to a second coupling, according to an embodiment.

FIGS. 4A and 4B are cross-sectional schematics illustrating a method of attaching a porous material to a second coupling, according to an embodiment.

FIG. 5 is a cross-sectional schematic of a porous material with a second coupling attached thereto, according to an embodiment.

FIG. 6 is a cross-sectional schematic of a porous material with a second coupling attached thereto, according to an embodiment.

FIG. 7 is a cross-sectional schematic of a second coupling according to an embodiment.

FIG. 8A is a top plan view of a second coupling that includes the fastener and the spine separated, according to an embodiment.

FIG. 8B is a cross-sectional schematic of the second coupling fully assembled, wherein the cross-sectional is taken along plane 8B-8B of the spine illustrated in FIG. 8A.

FIGS. 8C and 8D is cross-sectional schematics of different methods of attaching the spine to a porous material, according to different embodiments.

FIG. 9 is a cross-sectional schematic of a second coupling that defines one or more passageways extending therethrough, according to an embodiment.

FIG. 10A is a side view of a second coupling that is a clip, according to an embodiment.

FIG. 10B is a cross-sectional schematic of a fluid collection assembly that includes the clip, according to an embodiment.

FIG. 11 is a cross-sectional view of a fluid collection assembly including a fluid impermeable barrier defining a vent (i.e., aperture) at or near a proximal end region thereof, according to an embodiment.

FIG. 12 is an isometric view of a fluid collection assembly including a fluid impermeable barrier having an aperture formed in at least one lateral flange, according to an embodiment.

FIG. 13 is a front elevational view of a fluid collection assembly, according to an embodiment.

FIG. 14 is cross-sectional view of a fluid collection assembly, according to an embodiment.

FIG. 15 is a block diagram of a fluid collection system for fluid collection, according to an embodiment.

### DETAILED DESCRIPTION

Embodiments are directed to fluid collection assemblies including a fluid impermeable barrier defining at least one of a flattened sump or at least one aperture, fluid collection systems including the same, and methods of using and forming the same. An example fluid collection assembly includes a fluid impermeable barrier and at least one porous material disposed in the fluid impermeable barrier. The fluid impermeable barrier includes a proximal end region (i.e., a region that may be closest to a patient's navel during use) and a distal end region opposite the proximal end region. The fluid impermeable barrier defines a chamber, at least one opening that allows bodily fluids discharged by the patient to flow into the chamber, and a fluid outlet at or near the distal end region of the fluid impermeable barrier. The fluid impermeable barrier also defines at least one of a flattened sump at or near the distal end region or at least one aperture. The at least one aperture formed in the fluid impermeable barrier may form at least one of at least one first coupling configured to be attached to at least one second coupling that is attached to the porous material, at least one vent extending through the fluid impermeable barrier at or near the proximal end region thereof, or at least one indentation formed in at least one lateral flange extending laterally outwardly from portions of the fluid impermeable barrier defining the chamber.

During use, the fluid collection assembly may be positioned on patient (e.g., individuals using the fluid collection assembly) such that the porous material is positioned adjacent to a urethral opening of the patient. The patient may discharge one or more bodily fluids (e.g., urine, sweat, blood, etc.). The discharged bodily fluids may be received into the porous material. The bodily fluids may flow through the porous material to an inlet of a conduit positioned through a fluid outlet defined by the fluid impermeable barrier. The bodily fluids then may flow through the conduit to be removed from the fluid collection assembly. In an embodiment, a vacuum may be provided from the conduit to the porous material. The vacuum may facilitate flow of the bodily fluids through the porous material to the inlet of the conduit. The vacuum may also facilitate flowing the bodily fluids through the conduit. The vacuum may be provided from a vacuum source that is in fluid communication with the conduit.

As will be discussed in more detail below, the fluid collection assemblies disclosed herein may exhibit one or more improvements over conventional fluid collection assemblies. In an example, the fluid impermeable barriers of conventional fluid collection assemblies may be flimsy which makes securing porous material therein difficult unless adhesives are used. However, the adhesives may block or otherwise obstruct at least some of the pores in the porous material thereby inhibiting flow of the bodily fluids therethrough. The fluid collection assemblies disclosed herein may at least one first coupling and at least one second coupled that, when attached together, allow the porous material to be secured to the fluid impermeable barrier without using an adhesive. In an example, conventional fluid collection assemblies often have vents formed therein at or near the distal end regions thereof to facilitate air flow in the chamber. However, such locations of the vents in such conventional fluid collection assemblies increase the likelihood that bodily fluids leak from the conventional fluid collection assemblies since the bodily fluids flow towards and tend to pool near the fluid outlet. The fluid collection assemblies disclosed herein may include at least one vent formed in or near the proximal end region of the fluid impermeable barrier which has been found to still facilitate air flow through the chamber while minimizing the likelihood that the bodily fluids leak from the chamber. In an example, it has been found that old patients have difficultly holding (e.g., droping) and manipulating (e.g., bending) conventional fluid collection assemblies that include lateral flanges. The fluid collection assemblies disclosed herein, when including at least one lateral flange, may include at least one indentation formed in the lateral flange which help the patient hold the fluid collection assemblies (e.g., decrease the likelihood that the patient drops the fluid collection assembly) and helps the patient manipulate the fluid collection assembly. In an example, conventional fluid collection assemblies may include a positioning feature configured to be positioned adjacent to the vaginal opening of the patient to facilitate correct positioning of the conventional fluid collection assembly. However, it has been found that such positioning features may inadvertently penetrates into the vaginal opening which may cause patient discomfort. The fluid collection assemblies disclosed herein may include a flattened sump instead of the positioning features of conventional fluid collection assemblies. It has been found that the flattened sump facilitates positioning of the fluid collection assembly while decreasing the likelihood the that the flattened sump penetrates into the vaginal opening.

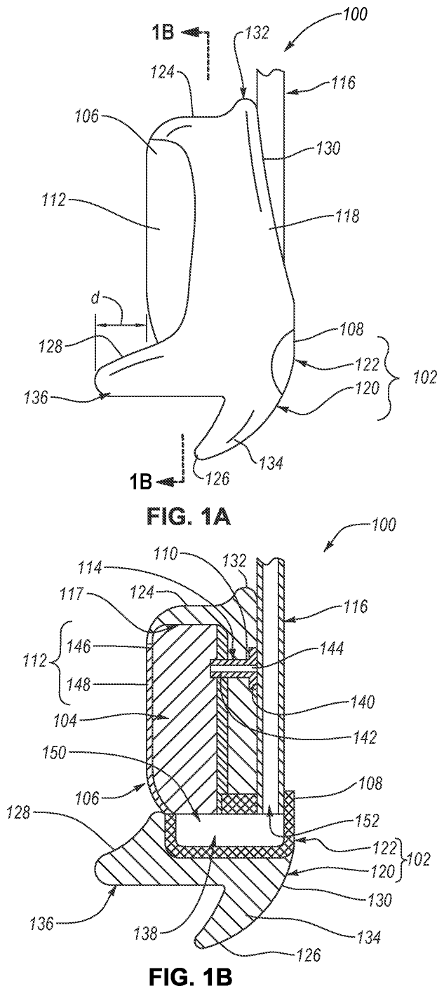

FIG. 1A is a side elevational view of a fluid collection assembly 100, according to an embodiment. FIG. 1B is a cross-sectional schematic of the fluid collection assembly 100 taken along plane 1B-1B, according to an embodiment. The fluid collection assembly 100 is example of a female fluid collection assembly for receiving and collecting bodily fluids from a female. The fluid collection assembly 100 includes a fluid impermeable barrier 102 that at least defines a chamber 104, at least one opening 106, and a fluid outlet 108. The fluid impermeable barrier 102 also includes at least one first coupling. In an example, as illustrated, the first coupling is an aperture 110 extending through the fluid impermeable barrier 102. The fluid collection assembly 100 also includes at least one porous material 112 positioned within the chamber 104. The fluid collection assembly 100 also includes at least one second coupling that is configured to be attached to the porous material 112 and interact with the first coupling. In an example, as illustrated, the second coupling includes a fastener 114 partially positioned through the aperture 110. The fluid collection assembly 100 may also include at least one conduit 116 partially positioned within the fluid outlet 108 that is configured to remove one or more bodily fluids from the chamber 104. The conduit 116 may not extend through the porous material 112.

The fluid impermeable barrier 102 at least partially defines a chamber 104 (e.g., interior region) and an opening 106. For example, the interior surface(s) 117 of the fluid impermeable barrier 102 at least partially defines the chamber 104 within the fluid collection assembly 100. The fluid impermeable barrier 102 temporarily stores the bodily fluids in the chamber 104. The fluid impermeable barrier 102 may be formed of any suitable fluid impermeable material(s), such as a fluid impermeable polymer (e.g., silicone, polypropylene, polyethylene, polyethylene terephthalate, neoprene, a polycarbonate, thermoplastic polymer, etc.), a metal film, natural rubber, another suitable material, any other fluid impermeable material disclosed herein, or combinations thereof. As such, the fluid impermeable barrier 102 substantially prevents the bodily fluids from passing through the fluid impermeable barrier 102. In an example, the fluid impermeable barrier 102 may be air permeable and fluid impermeable. In such an example, the fluid impermeable barrier 102 may be formed of a hydrophobic material that defines a plurality of pores. At least one or more portions of at least an outer surface 118 of the fluid impermeable barrier 102 may be formed from a soft and/or smooth material, thereby reducing chaffing.

The opening 106 provides an ingress route for bodily fluids to enter the chamber 104. The opening 106 may be defined by the fluid impermeable barrier 102 such as by an inner edge of the fluid impermeable barrier 102. For example, the opening 106 is formed in and extends through the fluid impermeable barrier 102, from the outer surface 118 to the inner surface 117, thereby enabling bodily fluids to enter the chamber 104 from outside of the fluid collection assembly 100.

The opening 106 may be an elongated hole in the fluid impermeable barrier 102. For example, the opening 106 may be defined as a cut-out in the fluid impermeable barrier 102. The opening 106 may be located and shaped to be positioned adjacent to a female urethral opening. The opening 106 may have an elongated shape because the space between the legs of a female is relatively small when the legs of the female are closed, thereby only permitting the flow of the bodily fluids along a path that corresponds to the elongated shape of the opening 106 (e.g., longitudinally extending opening).

The fluid collection assembly 100 may be positioned proximate to the female urethral opening and the bodily fluids may enter the chamber 104 of the fluid collection assembly 100 via the opening 106. The fluid collection assembly 100 is configured to receive the bodily fluids into the chamber 104 via the opening 106. When in use, the opening 106 may have an elongated shape that extends from a first location below the urethral opening (e.g., at or near the anus or the vaginal opening) to a second location above the urethral opening (e.g., at or near the top of the vaginal opening or the pubic hair).

In some examples, the fluid impermeable barrier 102 may define a fluid outlet 108 sized to receive the conduit 116. The at least one conduit 116 may be disposed in the chamber 104 via the fluid outlet 108. The fluid outlet 108 may be sized and shaped to form an at least substantially fluid tight seal against the conduit 116 or the at least one tube thereby substantially preventing the bodily fluids from escaping the chamber 104.

In an embodiment, the fluid impermeable barrier 102 may include a shell 120 and a connector piece 122 secured to the shell 120. The shell 120 of the fluid collection assembly 100 includes a proximal end region 124, a distal end region 126 opposite the proximal end region 124, a front side 128, and a back side 130 opposite the front side 128. Generally, during use, the distal end region 126 is closer to the gluteal cleft of the patient than the proximal end region 124 and the front side 128 generally faces the vaginal region of the patient. The shell 120 may be formed from silicone, neoprene, a thermoplastic elastomer, or other fluid impermeable material.

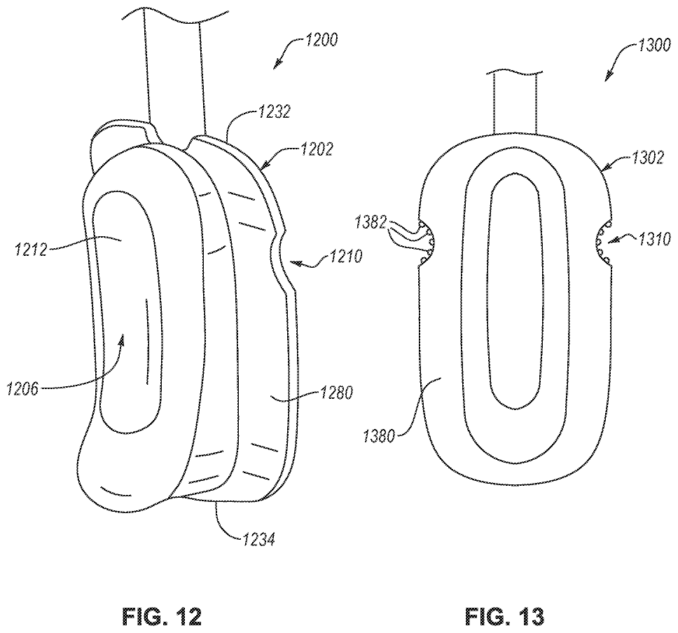

In an embodiment, the shell 120 includes one or more flanges. The flanges may provide more locations for underwear or other clothing to contact and press against the fluid collection assembly 100 which may facilitate securing the fluid collection assembly 100 to the vaginal region of the patient and may improve patient comfort. In an embodiment, the flanges may include at least one of an upper flange 132 forming the proximal end region 124 and a bottom flange 134 opposite the upper flange 132 that forms the distal end region 126. The flanges may also include at least one lateral flange (show in FIGS. 12 and 13) extending laterally outwardly from the portions of the fluid impermeable barrier 102 defining the chamber 104.

The flanges of the body may extend from the rest of the shell 120 by a distance that is about 1 mm or greater, about 1 mm or greater, about 3 mm or greater, about 4 mm or greater, about 5 mm or greater, about 6 mm or greater, about 7.5 mm or greater, about 1 cm or greater, about 1.25 cm or greater, about 1.5 cm or greater, about 1 cm or greater, about 1.5 cm or greater, about 3 cm or greater, about 4 cm or greater, about 5 cm or greater, or in ranges of about 1 mm to about 3 mm, about 1 mm to about 4 mm, about 3 mm to about 5 mm, about 4 mm to about 6 mm, about 5 mm to about 7.5 mm, about 6 mm to about 1 cm, about 7.5 mm to about 1.25 cm, about 1 cm to about 1.5 cm, about 1.25 cm to about 1 cm, about 1.5 cm to about 1.5 cm, about 1 cm to about 3 cm, about 1.5 cm to about 4 cm, or about 3 cm to about 5. The distance that the flanges extend from the rest of the shell 120 may be selected based on the expected size of the vaginal region of the patient (e.g., larger flanges for a larger vaginal region) or the expect rotational forces applied to the fluid collection assembly 100 during use. In some examples, at least some of the flanges may extend further from the rest of the shell 120 that other flanges. For instance, as illustrated, the bottom flange 134 may extend further from the rest of the fluid impermeable barrier 102 than the upper flange 132 since some patients may find the longer bottom flange 134 more comfortable.

In an embodiment, the one or more flanges may exhibit a concave curve relative to the front side 128 of the shell 120. The concave curve of the flanges may extend from the proximal end region 124 to the distal end region 126. The concave curve of the flanges may allow the flanges to better conform to the shape of the vaginal region since the vaginal region is curved. Conforming the flanges to the shape of the vaginal region may make the fluid collection assembly 100 more comfortable by more uniformly distributing pressure across the vaginal region, especially when the flanges contact the labia majora

In an embodiment, the shell 120 may include a sump 136 at or near the distal end region 126. The sump 136 may extend outwardly from the front side 128 of the shell 120 and/or the porous material 112 which facilitates positioning of the fluid collection assembly 100. For example, extending the sump 136 outwardly may cause the sump 136 to interface with (e.g., abut or partially penetrate) the vaginal opening of the patient. Such interfacing between the sump 136 and the vaginal opening may cause the fluid collection assembly 100 to be correctly positioned on the patient. It is noted that the sump 136 may exhibit a curved (e.g., not flattened) shape.

The sump 136 may extend outwardly from the front side 138 of the shell 120 and/or the porous material 112 by a distance d. The distance d may be about 1 mm to about 3 mm, about 2 mm to about 4 mm, about 3 mm to about 5 mm, about 4 mm to about 6 mm, about 5 mm to about 7 mm, about 6 mm to about 8 mm, about 7 mm to about 9 mm, about 8 mm to about 10 mm, about 9 mm to about 11 mm, about 10 mm to about 12 mm, about 11 mm to about 13 mm, about 12 mm to about 14 mm, or about 13 mm to about 15 mm. The distance d may be selected based on a number of factors. For example, increasing the distance d increases the likelihood that the sump 136 interfaces with the vaginal opening but also increases the likelihood that the sump 136 partially penetrates the vaginal opening or otherwise makes the fluid collection assembly 100 uncomfortable to wear.

During use, the sump 136 is configured to define portions of the chamber 104 that are at, near, or otherwise in fluid communication with a gravimetric low point of the porous material 112. For example, the portions of the chamber 104 defined by the sump 136 may receive a portion of the porous material 112 therein. The portions of the chamber 104 defined by the sump 136 may receive at least some of the bodily fluids that are received by the porous material 112. The sump 136 may prevent or at least inhibit bodily fluids from leaking from the fluid collection assembly 100. The sump 136 may include at least a portion of the connector piece 122 at least partially disposed therein.

In an embodiment, the shell 120 may define a recess that is configured to receive a conduit 116. The recess may extend from or near the proximal end region 124 to or near the distal end region 126 thereby allowing the conduit 116 to extend from or near the patient's abdominal region to the connector piece 122. In an embodiment, the recess may be configured such that the shell 120 encloses and/or abuts less than 50% of a circumference of the conduit 116, thereby allowing the conduit 116 to freely enter and leave the recess during use. Allowing the conduit 116 to freely enter and leave the recess may facilitate positioning of the fluid collection assembly 100 such that the porous material 112 is adjacent to the vaginal region even when the conduit 116 is bending away from the vaginal region. Also, allowing the conduit 116 to freely enter and leave the recess may increase the likelihood that movement of the conduit 116 does not move the porous material 112 relative to the vaginal region since movement of the porous material 112 may cause leaking. In an embodiment, at least a portion of the recess may be configured such that the shell 120 encloses and/or abuts more than 50% (e.g., 51% to about 55%, about 53% to about 57%, or about 55% to about 60%) of the circumference of the conduit 116. Enclosing more than 50% of the circumference of the conduit 116 may more securely attach the conduit 116 to the shell 120 and may allow the conduit 116 to provide additional structure to the shell 120. The percentage of the conduit 116 enclosed and/or abutted by the shell 120 may be selected such that the inherent elasticity of the shell 120 and the conduit 116 allows the conduit 116 to be easily snapped into and out of the recess. As such, the conduit 116 may be removed from the recess to facilitate positioning the porous material 112 adjacent to the vaginal region or when the conduit 116 is moved.

As previously discussed, the fluid impermeable barrier 102 includes a connector piece 122 that is attached to (e.g., with an adhesive, welding, interference fit, etc.) or integrally formed with the shell 120. The connector piece 122 is positioned at or near the distal end region 126 of the shell 120 which allows the connector piece 122 to receive bodily fluids that flow to the gravimetric low point of the porous material 112. In an embodiment, a portion of the connector piece 122 may be positioned in the sump 136 of the shell 120. In an embodiment, not shown, the connector piece 122 may form the sump 136 instead of the shell 120.

The connector piece 122 is configured to be connected (e.g., fluidly connected) to the conduit 116. As such, the connector piece 122 may define the fluid outlet 108 which configured to be attached to or otherwise in fluid communication with the conduit 116. The fluid outlet 108 may be positioned adjacent to the back side 130 of the fluid impermeable barrier 102. The connector piece 122 may also define a channel 138 (e.g., tube) configured to allow the porous material 112 and the sump 136 to be in fluid communication with the conduit 116.

In an embodiment, at least a portion of the connector piece 122 may exhibit a rigidity that is greater than the shell 120. The increased rigidity of the connector piece 122 relative to the shell 120 may facilitate attachment of the conduit 116 to the connector piece 122. In an example, the connector piece 122 may exhibit a rigidity that is greater than the shell 120 when the connector piece 122 is formed from a material exhibiting at least one of a greater Young's modulus (i.e., modulus of elasticity), yield strength, or ultimate tensile strength than a material that forms the shell 120. In an example, the connector piece 122 may exhibit a rigidity that is greater than the shell 120 when the connector piece 122 exhibits a thickness that is greater than the shell 120.

As previously discussed, the fluid impermeable barrier 102 includes a first coupling and a second coupling. The first and second couplings may allow the fluid collection assembly 100 to exhibit one or more improvements over conventional fluid collection assemblies. For example, the porous material 112 may be relatively flimsy (e.g., flexible or elastic), which makes securing the porous material 112 to the fluid impermeable barrier 102 difficult. For instance, merely disposing the porous material 112 in the chamber 104 defined by the fluid impermeable barrier 102 may not prevent the porous material 112 from falling out of the chamber 104 due to the flimsiness thereof. Conventional fluid collection assemblies often secure the porous material thereof to the fluid impermeable barrier thereof by extending the conduit into the chamber and positioning the conduit within porous material (e.g., the porous material defines a bore configured to receive the conduit). Positioning the conduit within the porous material helps maintain the porous material in the chamber since the conduit provides structure to the porous material. Disposing the conduit in the chamber, however, presents several issues. For example, generally, it can be preferable to cause the porous material 112 to exhibit a shape that corresponds to the shape of the region about the urethral opening of the patient to prevent bodily fluids from leaking. The conduit disposed in the chamber causes the porous material having the conduit positioned therein to exhibit a shape that corresponds to the shape of the conduit which may be different than the shape of the region about the urethral opening, especially since the shape of the region about the urethral opening may vary from patient to patient. Further, over-insertion of the conduit into the chamber may inhibit bodily fluids flowing into the conduit, for instance, when over-insertion of the conduit causes the inlet to press against the fluid impermeable barrier. Conventional fluid collection assemblies also often secure the porous material to the fluid impermeable barrier by adhesively attaching the porous material to the fluid impermeable barrier instead of or in addition to using the conduit to secure the porous material to the fluid impermeable barrier. However, using an adhesive to secure the porous material to the fluid impermeable barrier complicates manufacturing by requiring an additional manufacturing step of applying an adhesive to at least one of the porous material or the fluid impermeable barrier and may make automation of the manufacturing of the fluid collection assembly more difficult. Further, the porous material tends to absorb or otherwise receive the adhesive which obstructs the passages of the porous material through which the bodily fluids may flow.

The fluid collection assembly 100 uses the first and second couplings to remedy these issues of using a conduit and/or adhesive to secure the porous material to the fluid impermeable barrier. In particular, the first and second couplings interact with each other to secure the porous material 112 to the fluid impermeable barrier 102. In an example, the first coupling of the fluid impermeable barrier includes at least one aperture 110 formed therein (i.e., an aperture formed in the fluid impermeable barrier 102). The fluid impermeable barrier may already have the aperture 110 formed therein since fluid impermeable barriers often define perforations therein as vacuum relief openings and, as such, selecting the first coupling to be an aperture 110 allows little to not modifications to fluid impermeable barrier. In an example, as illustrated, the second coupling may include at least one fastener 114 that is partially positioned through the aperture 110 and secured to the porous material. It is noted that, as used herein, the first and second couplings may not include an adhesive or the conduit that removes bodily fluids from the chamber.

The first coupling may include an aperture 110 extending through the fluid impermeable barrier 102. In an embodiment, as illustrated, the aperture 110 may be formed in the shell 120. For instance, the aperture 110 may be formed in the shell 120 at or near the proximal end region 124. Attaching the porous material 112 at or near the proximal end region 124 may allow the porous material 112 to dangle from the first coupling during use, which may decrease the likelihood that a portion of the porous material 112 falls out of the chamber 104 than if the aperture 110 is formed at or near the distal end region of the shell 120. In an embodiment, the aperture 110 may be formed at least one of at or near the distal end region 126 of the shell, in a middle of the shell 120, or in the connector piece 122. It is noted that the first coupling may include structures other than or in addition to the aperture 110. For example, the first coupling may include a loop or hook extending from the fluid impermeable barrier 102, a hoop-and-loop structure (e.g., Velcro), a perforation only extending partially through the fluid impermeable barrier 102, or any other suitable structure.

As previously discussed, the fluid collection assembly 100 includes a second coupling. In an embodiment, as illustrated, the second coupling is a fastener 114 (e.g., a bolt). The fastener 114 includes a head 140 and a rod or shaft 142 extending from the head 140. At least a portion of the rod 142 is configured to be attached to the porous material 112. For example, at least a portion of the rod 142 opposite the head 140 is attached to the porous material 112 using an adhesive, a weld (e.g., ultrasonic weld), a threaded attachment (e.g., the rod 142 includes threads that engage the fibers of the porous material 112), or any other attachment technique.

The head 140 and the rod 142 exhibit lateral dimensions that are configured to substantially prevent the head 140 from moving through the aperture 110 and allow the rod 142 to pass through the aperture 110. For example, the head 140 may exhibit a lateral dimension (e.g., maximum lateral dimension) that is greater than a corresponding lateral dimension of the rod 142. The lateral dimension of the head 140 may be larger than a corresponding lateral dimension of a portion of the aperture 110. The larger lateral dimension of the head 140 compared to the corresponding lateral dimension of the aperture 110 may inhibit the head 140 passing completely through the aperture 110. In other words, the larger lateral dimension of the head 140 compared to the corresponding lateral dimension of the aperture 110 may form an obstacle to the head 140 passing through the aperture 110. However, it is noted that the fluid impermeable barrier 102, especially the shell 120, may be formed from an elastic material which allows the head 140 to be forced through the aperture 110 upon an application of a force sufficiently large to deform the fluid impermeable barrier 102. However, the force required to force the head 140 through the fluid impermeable barrier 102 is sufficiently large that the head 140 is unlikely to pass through the fluid impermeable barrier 102 during use. The lateral dimension of the rod 142 is smaller than or substantially equal to the corresponding lateral dimension of the aperture 110 thereby allowing the rod 142 to pass through the aperture 110. As such, the rod 142 may pass through one side of the fluid impermeable barrier 102 to the other side. Attaching the rod 142 to the porous material 112, in combination with the head 140, substantially prevents the fastener 114 from passing through the aperture 110.

In an embodiment, the aperture 110 is configured such that the head 140 is not positioned therein. In such an embodiment, the head 140 may protrude from the fluid impermeable barrier 102 which may make the fluid collection assembly 100 more uncomfortable to use and may push the conduit 116 away from the fluid impermeable barrier 102. In an embodiment, the aperture 110 is configured such that the head 140 is positioned in a portion thereof. In such an embodiment, the aperture 110 may include a first portion and a second portion. The first portion may be configured to have at least a portion of the head 140 positioned therein. For example, the first portion may exhibit a lateral dimension that is equal to or slightly larger than the corresponding lateral dimension of the head 140. Positioning the head 140 in the first portion may prevent the head 140 from extending from the fluid impermeable barrier 102 or at least decrease the distance that the head 140 extends from the fluid impermeable barrier 102, either of which may make using the fluid collection assembly 100 more comfortable and may allow the conduit 116 to be positioned adjacent to the fluid impermeable barrier 102. The second portion of the aperture 110 may be configured to prevent or at least inhibit the head 140 from passing therethrough. For example, the second portion of the aperture 110 may exhibit a lateral dimension that is smaller than the head 140.

As previously discussed, the aperture 110 may form a vacuum relief opening when the fastener 114 is not positioned therethrough. The vacuum relief opening allows air to enter the chamber 104 which improves fluid flow through the porous material 112 and prevents the fluid collection assembly 100 from being suctioned to the patient which may cause general discomfort. Positioning the fastener 114 through the aperture 110 may obstruct the vacuum relief opening such that air cannot enter the chamber 104 through the aperture 110. As such, in some embodiments, the fastener 114 may define a passageway 144 extending therethrough. For example, the passageway 144 may extend from the head 140 to a portion of the rod 142 that is not obstructed by the fluid impermeable barrier 102 (e.g., extend centrally from the head 140 to the rod 142). The passageway 144 allows air to flow through the fastener 114 and into the chamber 104. It is noted that the fluid impermeable barrier 102 may define a vacuum relief opening that is spaced from and not at least partially occupied by the fastener 114.

As previously discussed, the porous material 112 is disposed in the chamber 104. The porous material 112 may cover at least a portion (e.g., all) of the opening 106. The porous material 112 may include a fluid permeable membrane 146 and a fluid permeable support 148. The porous material 112 is exposed to the environment outside of the chamber 104 through the opening 106. In an embodiment, the porous material 112 may be configured to wick any bodily fluids away from the opening 106, thereby preventing the bodily fluids from escaping the chamber 104. The permeable properties referred to herein may be wicking, capillary action, diffusion, or other similar properties or processes, and are referred to herein as “permeable” and/or “wicking.” Such “wicking” and/or “permeable” properties may not include absorption of the bodily fluids into at least a portion of the porous material 112, such as not include adsorption of the bodily fluids into the fluid permeable support 148. Put another way, substantially no absorption or solubility of the bodily fluids into the material may take place after the material is exposed to the bodily fluids and removed from the bodily fluids for a time. While no absorption or solubility is desired, the term “substantially no absorption” may allow for nominal amounts of absorption and/or solubility of the bodily fluids into the porous material 112 (e.g., absorbency), such as less than about 30 wt % of the dry weight of the porous material 112, less than about 10 wt %, less than about 10 wt %, less than about 7 wt %, less than about 5 wt %, less than about 3 wt %, less than about 1 wt %, less than about 1 wt %, or less than about 0.5 wt % of the dry weight of the porous material 112. The porous material 112 may also wick the bodily fluids generally towards an interior of the chamber 104, as discussed in more detail below. In an embodiment, the porous material 112 may include at least one absorbent or adsorbent material.

In an embodiment, the porous material 112 may include the fluid permeable membrane 146 disposed in the chamber 104. The fluid permeable membrane 146 may cover at least a portion (e.g., all) of the opening 106. The fluid permeable membrane 146 may be composed to wick the bodily fluids away from the opening 106, thereby preventing the bodily fluids from escaping the chamber 104.

The fluid collection assembly 100 may include the fluid permeable support 148 disposed in the chamber 104. The fluid permeable support 148 is configured to support the fluid permeable membrane 146 since the fluid permeable membrane 146 may be formed from a relatively foldable, flimsy, or otherwise easily deformable material. For example, the fluid permeable support 148 may be positioned such that the fluid permeable membrane 146 is disposed between the fluid permeable support 148 and the fluid impermeable barrier 102. As such, the fluid permeable support 148 may support and maintain the position of the fluid permeable membrane 146. The fluid permeable support 148 may include any material that may wick, absorb, adsorb, or otherwise allow fluid transport of the bodily fluids, such as any of the fluid permeable membrane materials disclosed herein above. For example, the fluid permeable membrane material(s) may be utilized in a more dense or rigid form than in the fluid permeable membrane 146 when used as the fluid permeable support 148. The fluid permeable support 148 may be formed from any fluid permeable material that is less deformable than the fluid permeable membrane 146. For example, the fluid permeable support 148 may include a porous polymer (e.g., nylon, polyester, polyurethane, polyethylene, polypropylene, etc.) structure or an open cell foam, such as spun nylon fiber. In some examples, the fluid permeable support 148 may include a nonwoven material. In some examples, the fluid permeable support 148 may be formed from a natural material, such as cotton, wool, silk, or combinations thereof. In such examples, the material may have a coating to prevent or limit absorption of fluid into the material, such as a water repellent coating. In some examples, the fluid permeable support 148 may be formed from fabric, felt, gauze, or combinations thereof.

In an embodiment, the fluid permeable membrane 146 may include any material that may wick the bodily fluids. For example, the fluid permeable membrane 146 may include fabric, such as a gauze (e.g., a silk, linen, or cotton gauze), another soft fabric, another smooth fabric, a nonwoven material, or any of the other porous materials disclosed herein. Forming the fluid permeable membrane 146 from gauze, soft fabric, and/or smooth fabric may reduce chaffing caused by the fluid collection assembly 100. In some examples, the fluid permeable membrane 146 may be optional. For example, the porous material 112 may include only the fluid permeable support 148. In some examples, the fluid permeable support 148 may be optionally omitted from the fluid collection assembly 100. For example, the porous material 112 may only include the fluid permeable membrane 146.

In an embodiment, at least a portion of the porous material 112 (e.g., one or more of the fluid permeable membrane 146 or, more preferably, the fluid permeable support 148) may be hydrophobic. The porous material 112 may be hydrophobic when the porous material 112 exhibits a contact angle with water (a major constituent of bodily fluids) that is greater than about 90°, such as in ranges of about 90° to about 120°, about 105° to about 135°, about 120° to about °, about 135° to about 175°, or about ° to about 180°. The hydrophobicity of the porous material 112 may limit absorption, adsorption, and solubility of the bodily fluids in the porous material 112 thereby decreasing the amount of bodily fluids held in the porous material 112. In an embodiment, at least a portion of the porous material 112 is hydrophobic or hydrophilic. In an embodiment, the fluid permeable support 148 is more hydrophobic (e.g., exhibits a larger contact angle with water) than the fluid permeable membrane 146. The lower hydrophobicity of the fluid permeable membrane 146 may help the porous material 112 receive the bodily fluids from the urethral opening while the hydrophobicity of the fluid permeable support 148 limits the bodily fluids that are retained in the porous material 112.

The porous material 112 may at least substantially completely fill the portions of the chamber 104 that are not occupied by the conduit 116 or the connector piece 122. In some examples, the porous material 112 may not substantially completely fill the portions of the chamber 104 that are not occupied by the conduit 116 and the connector piece 122. In such an example, the fluid collection assembly 100 includes the reservoir disposed in the chamber 104. The bodily fluids that are in the chamber 104 may flow through the fluid permeable membrane 146 and/or fluid permeable support 148 to the reservoir. The reservoir may retain of the bodily fluids therein. The reservoir may be located in a portion of the chamber 104 that is designed to be located in a gravimetrically low point of the fluid collection assembly when the fluid collection assembly is worn.

The conduit 116 may be at least partially disposed in the chamber 104. The conduit 116 may be used to remove the bodily fluids from the chamber 104. The conduit 116 includes at least one wall defining an inlet 152 and an outlet (not shown) downstream from the inlet 152. The outlet of the conduit 116 may be operably coupled to a vacuum source, such as a vacuum pump for withdrawing fluid from the chamber 104 through the conduit 116. For example, the conduit 116 may extend into the fluid impermeable barrier 102 from the proximal end region 124 and may extend to the distal end region 126 to a point proximate to the reservoir therein such that the inlet 152 is in fluid communication with the reservoir. The conduit 116 fluidly couples the chamber 104 with the fluid storage container (not shown) or the vacuum source (not shown).

In an embodiment, the conduit 116 extends from the fluid outlet 108 to a location that is proximate to the reservoir. In an embodiment, the conduit 116 is at least partially disposed in the reservoir and the inlet 152 may be extended into or be positioned in the reservoir. The bodily fluids collected in the fluid collection assembly 100 may be removed from the chamber 104 via the conduit 116.

Locating the inlet 152 at or near a location expected to be the gravimetrically low point of the chamber 104 when worn by an patient enables the conduit 116 to receive more of the bodily fluids than if inlet 152 was located elsewhere and reduce the likelihood of pooling (e.g., pooling of the bodily fluids may cause microbe growth and foul odors). For instance, the bodily fluids in the fluid permeable membrane 146 and the fluid permeable support 148 may flow in any direction due to capillary forces. However, the bodily fluids may exhibit a preference to flow in the direction of gravity, especially when at least a portion of the fluid permeable membrane 146 and/or the fluid permeable support 148 is saturated with the bodily fluids. Accordingly, one or more of the inlet 152 or the reservoir may be located in the fluid collection assembly 100 in a position expected to be the gravimetrically low point in the fluid collection assembly 100 when worn by a patient, such as the distal end region 126.

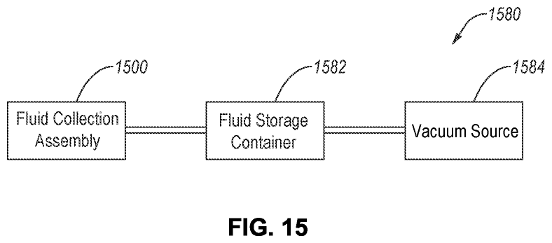

The inlet 152 and the outlet of the conduit 116 are configured to fluidly couple (e.g., directly or indirectly) the vacuum source (not shown) to the chamber 104 (e.g., the reservoir). As the vacuum source (FIG. 15) applies a vacuum/suction in the conduit 116, the bodily fluids in the chamber 104 (e.g., at the distal end region 126 such as in the reservoir) may be drawn into the inlet 152 and out of the fluid collection assembly 100 via the conduit 116. In some examples, the conduit 116 may be frosted or opaque (e.g., black) to obscure visibility of the bodily fluids therein.

As previously discussed, the conduit 116 may be configured to be at least insertable into the chamber 104. In an example, the conduit 116 may be positioned in the chamber 104 such that a terminal end of the conduit 116 is spaced from the fluid impermeable barrier 102 or other components of the fluid collection assembly 100 that may at least partially obstruct or block the inlet 152. Further, the inlet 152 of the conduit 116 may be offset relative to a terminal end of the porous material 112 such that the inlet 152 is closer to the proximal end region 124 of the fluid collection assembly 100 than the terminal end of the porous material 112. Offsetting the inlet 152 in such a manner relative to the terminal end of the porous material 112 allows the inlet 152 to receive bodily fluids directly from the porous material 112 and, due to hydrogen bonding, pulls more bodily fluids from the porous material 112 into the conduit 116.

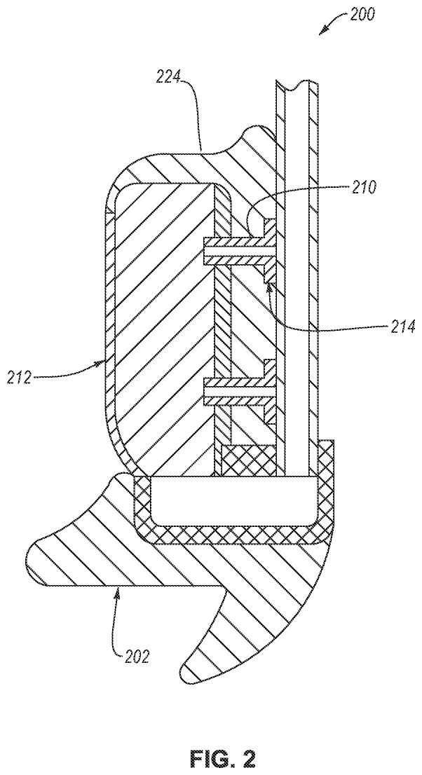

The fluid collection assembly 100 includes only a single first coupling and a single second coupling. However, the fluid collection assemblies disclosed herein may include a plurality of first couplings and a plurality of second couplings. For example, FIG. 2 is a cross-sectional schematic of a fluid collection assembly 200, according to an embodiment. Except as otherwise disclosed herein, the fluid collection assembly 200 is the same or substantially similar to any of the fluid collection assemblies disclosed herein. For instance, the fluid collection assembly 200 may include a fluid impermeable barrier 202 and at least one porous material 212.

The fluid impermeable barrier 202 includes a plurality of first couplings and the porous material 212 includes a plurality of second couplings. In the illustrated embodiment, the fluid impermeable barrier 202 includes a plurality of apertures 210 and the porous material 212 includes a plurality of fasteners 214 positioned through the apertures 210. In an example, the fluid impermeable barrier 202 includes a two apertures 210 and the porous material 212 includes two fasteners 214. In such an example, one of the apertures 210 may be located at or near the proximal end region 224 and the other perforation may be locate near an end of the porous material 212 opposite the proximal end region 224. As such, the apertures 210 and the fasteners 214 anchor opposing ends of the porous material 212 to the fluid impermeable barrier 202 which may more stably secure the porous material 212 to the fluid impermeable barrier 202 than if the apertures 210 were otherwise located.

The fasteners illustrated in FIGS. 1A-2 are illustrated as being directly attached to the porous materials. However, a patient using the fluid collection assemblies illustrated in FIGS. 1A-2 may feel the fasteners through the porous material, especially when the patient is sitting. Feeling the fasteners through the porous material may cause the patient discomfort since the pressure caused by the fasteners is not distributed across a large surface area. As such, in some embodiments, the fasteners may indirectly attached to the porous material via a spine.

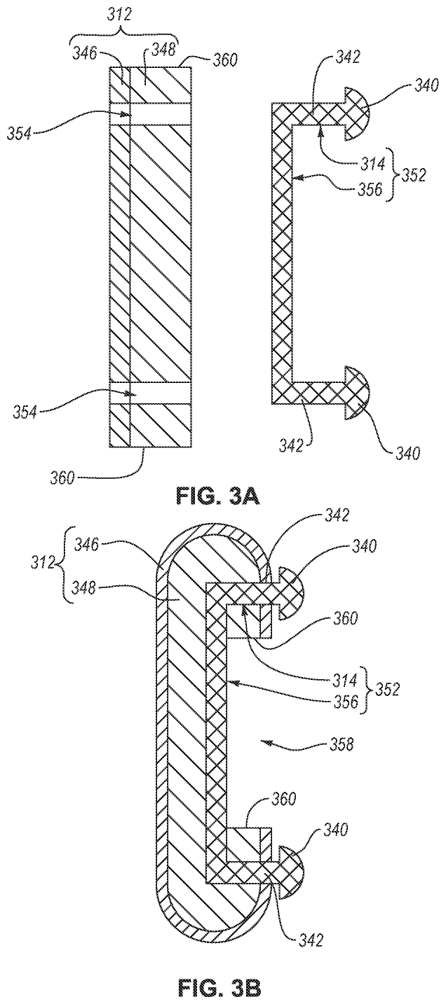

FIGS. 3A and 3B are cross-sectional schematics illustrating a method of securing a porous material 312 to a second coupling 352, according to an embodiment. Except as otherwise disclosed therein, the porous material 312 and the second coupling 352 may be the same or substantially similar to any of the porous materials and second couplings disclosed herein, respectively, and may be used in any of the fluid collection assemblies disclosed herein. For example, the porous material 312 may include a fluid permeable membrane 346 and a fluid permeable support 348 and the second coupling 352 may include a fastener 314 that includes a head 340 and a rod 342. It is noted the porous material 312 is illustrates as having the fluid permeable membrane 346 being disposed on only one side of the fluid permeable support 348. However, it is noted that the fluid permeable membrane 346 may enclose the circumference of the fluid permeable support 348, as illustrated in at least FIGS. 1B, 2, 8C, and 8D.

The porous material 312 defines a plurality of passageways 354 extending therethrough. For example, the passageways 354 may extend through the fluid permeable membrane 346 and the fluid permeable support 348. The passageways 354 are configured to have the rod 342 of the fastener 314 disposed therein. As such, the porous material 312 may define as many passageways 354 as the second coupling 352 includes fasteners 314. The passageways 354 may exhibit a size and shape that generally corresponds to the size and shape of the rod 342. However, the passageways 354 may exhibit a size and/or shape that inhibits the head 340 from moving therethrough. However, it is noted that the porous material 312 may exhibit sufficient elasticity that the head 340 may be forced through a corresponding passageway 354 substantially without damaging the porous material 312 when a sufficiently large force is applied to the fastener 314 thereby allowing the rod 342 to be positioned in the passageway 354. It is noted that the required to force the head 340 through the passageways 354 is greater than an expected force applied to the fastener 314 during use.

The second coupling 352 includes an elongated spine 356. The fasteners 314 of the second coupling 352 are attached to the spine 356 and the spine 356 may extend longitudinally between the fasteners 314. The spine 356 may provide structure to the second coupling 352. For example, the spine 356 may maintain a selected distance between the fasteners 314. The distance that the spine 356 maintains the fasteners 314 apart may help maintain the bend formed in the porous material 312 (as will be discussed in connection with FIG. 3B) and may facilitate attaching the second coupling 352 to the shell (not shown). The spine 356 may also make using the fluid collection assembly more comfortable. For example, as previously discussed, an patient using the fluid collection assemblies 100, 200 illustrated in FIGS. 1A-2 may be able to feel the fasteners (more particularly the rods) through the porous materials thereof, such as when the patient is sitting. The pressure caused by the fasteners, as felt through the porous materials, may make the fluid collection assemblies uncomfortable. However, the inclusion of the spine 356 to the second coupling 352 distributes the pressure caused by the fasteners over a larger area thereby making the fluid collection assembly more comfortable to use.

The spine 356 may also provide structure to the porous material 312 and at least a portion of the fluid impermeable barrier to which the second coupling 352 is attached. For example, the spine 356 may cause the porous material 312 and at least a portion of the fluid impermeable barrier to which the second coupling 352 is attached to exhibit a shape that generally corresponds to the shape of the spine 356. In an embodiment, the spine 356 may be formed from a shape memory material. As used herein, a shape memory material includes any material that may be shaped in response to a force (e.g., a chemical force, a physical force, heat, etc.) and maintain the shape. Examples of shape memory materials include nitinol, copper, steel, or aluminum. When the spine 356 is formed from a shape memory material, the spine 356 may exhibit a first shape. The porous material 312 and at least a portion of the fluid impermeable barrier may exhibit a shape that corresponds to the first shape of the spine 356. The spine 356 may be bent from the first shape to a second shape that is different than the first shape. The spine 356 may maintain the second shape because the spine 356 is formed from a shape memory material. Bending the spine 356 into the second shape may cause the porous material 312 and at least a portion of the fluid impermeable barrier to exhibit a shape that generally corresponds to the second shape of the spine 356. The spine 356 may be bent from the first shape to the second shape when the porous material 312 and at least a portion of the fluid impermeable barrier does not exhibit a shape that generally corresponds to the shape of the vaginal region of the patient using the fluid collection assembly. Bending the spine 356 to the second shape may cause the porous material 312 and at least a portion of the fluid impermeable barrier to exhibit a shape that better corresponds to the shape of the vaginal region of the patient. Causing the porous material 312 and at least a portion of the fluid impermeable barrier to exhibit a shape that generally corresponds to the shape of the vaginal region may inhibit bodily fluids leaking from the fluid collection assembly and/or cause the porous material 312 to receive a greater percentage of the bodily fluids discharged by the patient. In an embodiment, the spine 356 is formed from an elastic material (i.e., not brittle material) such that the spine 356 may be shaped or bent as needed without breaking or otherwise failing.

In an embodiment, as illustrated, the fasteners 314 may be integrally formed with the spine 356. For example, the second coupling 352 may be formed using an injection molding technique, 3D printing, or another suitable technique. Integrally forming the fasteners 314 and the spine 356 may minimize the number of manufacturing steps required to form the second coupling 352 since the fasteners 314 and the spine 356 do not need to be attached together.

It is noted that, unlike the fasteners 114 illustrated in FIG. 3B, the head 340 is illustrated as being curved. Curving the head 340 may facilitate pushing the head 340 through the porous material 312 and through the shell. However, unlike the generally flat heads illustrated in FIG. 1B, the curved head 340 may not lie flush against the shell. It is noted that the head 340 may exhibit a tapered, oblong, conical, or semispherical shape since these shape (and other similar shapes) may facilitate pushing the head 340 through the porous material 312 and through the shell.

The porous material 312 may exhibit a length that is greater than the length of the second coupling 352 prior to attaching the porous material 312 to the second coupling 352. Referring to FIG. 3B, the longer length of the porous material 312 allows the porous material 312 to bend around the second coupling. The fasteners 314 may be positioned through the passageways 354 defined by the porous material 312. For example, the head 340 of the fasteners 314 may be forced through the passageways 354 such that the rod 340 is positioned within the passageway 354. Positioning the fasteners 314 through the passageways 354 attaches the second coupling 352 to the porous material 312.

In an embodiment, after attaching the porous material 312 to the second coupling 352, a gap 358 may be formed between the terminal edges 360 (e.g., the top and bottom edges) of the porous material 312. The gap 358 may increase the volume of bodily fluids that may be temporarily stored in the chamber defined by the fluid impermeable barrier. However, the gap 358 may allow the bodily fluids to pool in the chamber which may cause leaking of the bodily fluids and/or encourage growth of organisms in the chamber. As such, in some embodiments, the gap 358 may be eliminated or at least reduced in size. In an example, the porous material 312 may exhibit a length that is sufficient that the terminal edges 360 abut each other after bending the porous material 312 and attaching the porous material 312 to the fasteners 314 (e.g., the porous material 312 exhibits a length greater than what is illustrated in FIGS. 3A and 3B). In an example, the porous material 312 may include an additional porous material that is configured to fit within and at least partially fill the gap 358.

In an embodiment, the spine 356 is not directly attached to the porous material 312. In such an embodiment, the spine 356 is attached to the porous material 312 indirectly through the fasteners 314 positioned through the passageways 354. In an embodiment, the spine 356 is directly attached to the porous material 312, which may cause the porous material 312 to exhibit a shape that more closely conforms the shape of the spine 356. In an example, the spine 356 is adhesively attached to the porous material 312. However, adhesively attaching the spine 356 to the porous material 312 may cause the adhesive to be absorbed into the porous material 312 before the adhesive solidifies which causes the adhesive to at least partially block at least some of the passageways through the porous material 312. In an example, the spine 356 is welded (e.g., ultrasonically welded) to the porous material 312 which resolves at least some of the issues caused by adhesively attaching the spine 356 to the porous material 312. When the spine 356 is welded to the porous material 312, the spine 356 may be at least partially formed from a weldable material, such as acrylonitrile butadiene styrene (“ABS”), a ABS-polycarbonate blend or copolymer, polyethylene, polypropylene, polycarbonate, or another suitable material. In other words, the spine 356 is not formed from a non-weldable material, such as silicone, a polythermal elastomer, or a thermoplastic. When the spine 356 includes a shape memory material that is non-weldable to the porous material 312, the spine 356 may include a coating of a weldable material over at least a portion of the shape memory material. In an example, the spine 356 may be sewn or otherwise attached to the porous material 312, though, it is noted, that sewing or otherwise attaching the spine 356 to the porous material 312 during an automated manufacturing process may be more difficult than welding the spine 356 to the porous material 312.

It is noted that the fasteners 314 may also be attached to the porous material 312 using an attachment technique in addition to positioning the fasteners 314 through the porous material 312. For example, the fasteners 314 may be adhesively attached, welded (since the fasteners 314 may be at least partially formed from the same weldable material as the spine 356), sewn, or otherwise attached to the porous material 312.

In an example, the rod 340 may exhibit a length that is greater than a thickness of the porous material 312 which allows a portion of the rod 340 to extent outwardly from the porous material 312 without compressing the porous material 312. The portion of the rod 340 extending outwardly from the porous material 312 may be positioned within the first coupling (e.g., through the perforation).

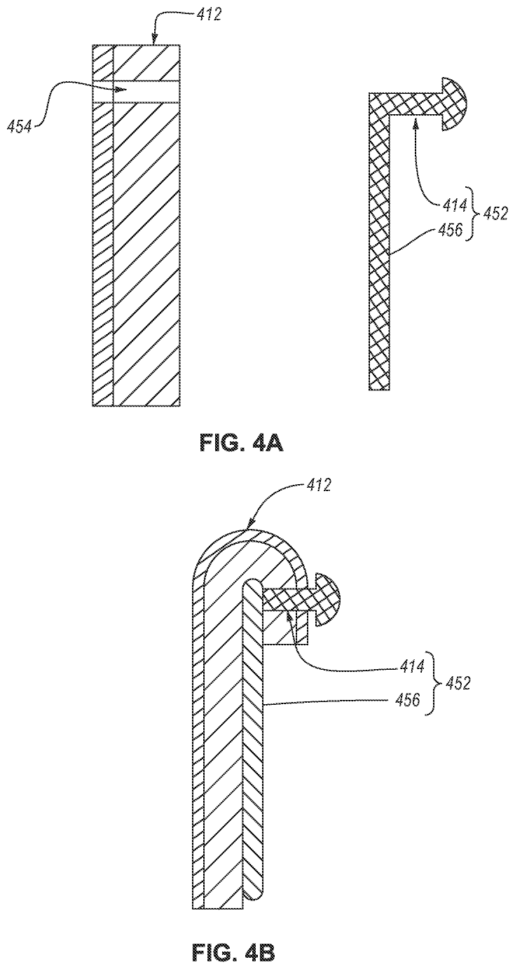

The porous material 312 and the second coupling 352 may be attached to the shell 220 illustrated in FIG. 2 since the shell 220 may be attached using a plurality of fasteners. However, the porous materials and second couplings including a spine may be configured to be attached to the shell 120 illustrated in FIG. 1B. For example, FIGS. 4A and 4B are cross-sectional schematics illustrating a method of attaching a porous material 412 to a second coupling 452, according to an embodiment. Referring to FIG. 4A, a porous material 412 and the second coupling 452 are provided. The porous material 412 and the second coupling 452 may be the same as the porous material 312 and the second coupling 352 illustrated in FIGS. 3A and 3B except that the porous material 412 only defines a single passageway 454 and/or the second coupling 452 includes a single fastener 414. Referring to FIG. 4B, the porous material 412 may be bent around a portion of the second coupling 452 such that the fastener 414 is positioned within the passageway 454. Even though only one fastener 414 is positioned through the porous material 412, the second coupling 452 (e.g., the spine 456 of the second coupling 452) provides structure to the porous material 412, may cause the porous material 412 to exhibit certain shape, and may make using the fluid collection assembly including the porous material 412 more comfortable to use.

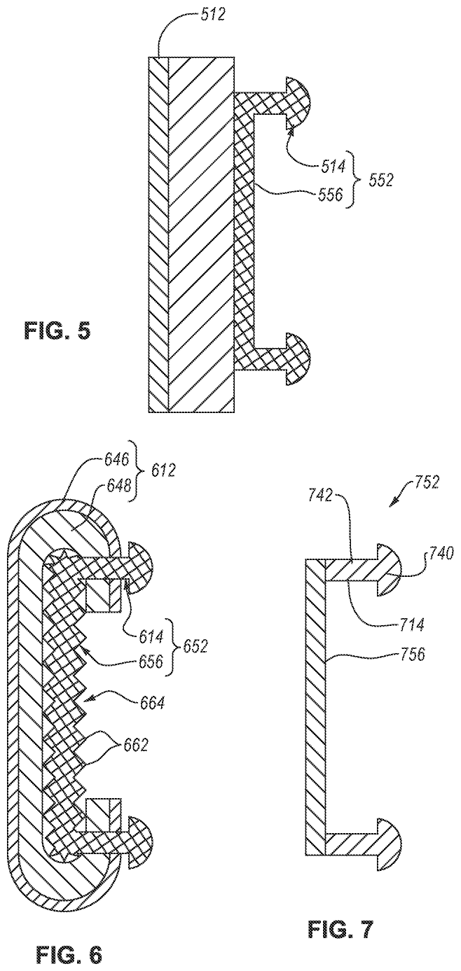

FIG. 5 is a cross-sectional schematic of a porous material 512 with a second coupling 552 attached thereto, according to an embodiment. Except as otherwise disclosed the porous material 512 and the second coupling 552 are the same or substantially similar to any of the porous materials and second couplings disclosed herein, respectively, and may be used in any of the fluid collection assemblies disclosed herein. However, unlike the other porous materials and second couplings disclosed herein, the porous material 512 does not define at least one aperture and/or the fasteners 514 of the second attachment are not positioned through the aperture. Instead, the spine 556 is directly attached to the porous material 512 (e.g., attached to a surface of the porous material 512 that is opposite the opening), for example, using an adhesive, welding, sewing, or another suitable technique.

FIG. 6 is a cross-sectional schematic of a porous material 612 with a second coupling 652 attached thereto, according to an embodiment. Except as otherwise disclosed herein, the porous material 612 and the second coupling 652 are the same as any of the porous materials and second couplings disclosed herein, respectively, and may be used in any of the fluid collection assemblies disclosed herein. For example, the porous material 612 may include a fluid permeable membrane 646 and a fluid permeable support 648 and the second coupling 652 may include one or more fasteners 614 and a spine 656.

The spine 656 exhibits a “crinkle” or sawtooth structure. The crinkle structure of the spine 656 includes one or more circumferentially or helically extending ridges 662 and defines one or more circumferentially or helically extending valleys 664 positioned between the ridges 662. The ridges 662 exhibit a wider lateral dimension (e.g., diameter) than portions of the spine 656 that define the valleys 664. The crinkle structure of the spine 656 may facilitate shaping of the spine 656 and, when the spine 656 is formed from a shape memory material, may facilitate maintaining the shape of the spine 656. For example, the crinkle structure of the spine 656 allows the spine 656 to bend by decreasing the distance between adjacent ridges 662. The valleys 664 provide a location for the ridges 662 to move into when the distance between adjacent ridges 662 decreases.

The second couplings illustrated in FIGS. 3A-6 illustrate the fasteners and the spines being integrally formed together. However, it is noted that the second couplings disclosed herein may be formed from two or more distinct components that are attached together. Forming the second coupling from two or more distinct components may allow the second coupling to be formed using more manufacturing techniques than if the second coupling was formed from a single piece. For example, FIG. 7 is a cross-sectional schematic of a second coupling 652 according to an embodiment. Except as otherwise disclosed herein, the second coupling 652 may be the same or substantially similar to any of the second coupling disclosed herein and may be used in any of the fluid collection assemblies disclosed herein. The second coupling 652 is formed from two or more distinct components that are attached together. In an embodiment, as illustrated, the second coupling 652 includes one or more fasteners 614 and a spine 656 that are distinct and attached from each other. In such an embodiment, the fasteners 614 and the spine 656 may be attached together using an adhesive, welding (e.g., ultrasonic welding), threadedly attaching, or any other suitable technique. When the fasteners 614 and the spine 656 are attached together using ultrasonic welding, the fasteners 614 and the spine 656 may be formed from a weldable material, as previously discussed. In an embodiment, one or more of the fasteners 614 are formed from distinct components (e.g., the head 640 and the rod 642 are distinct) that are attached together. In an embodiment, the spine 656 may be formed from two or more distinct components.

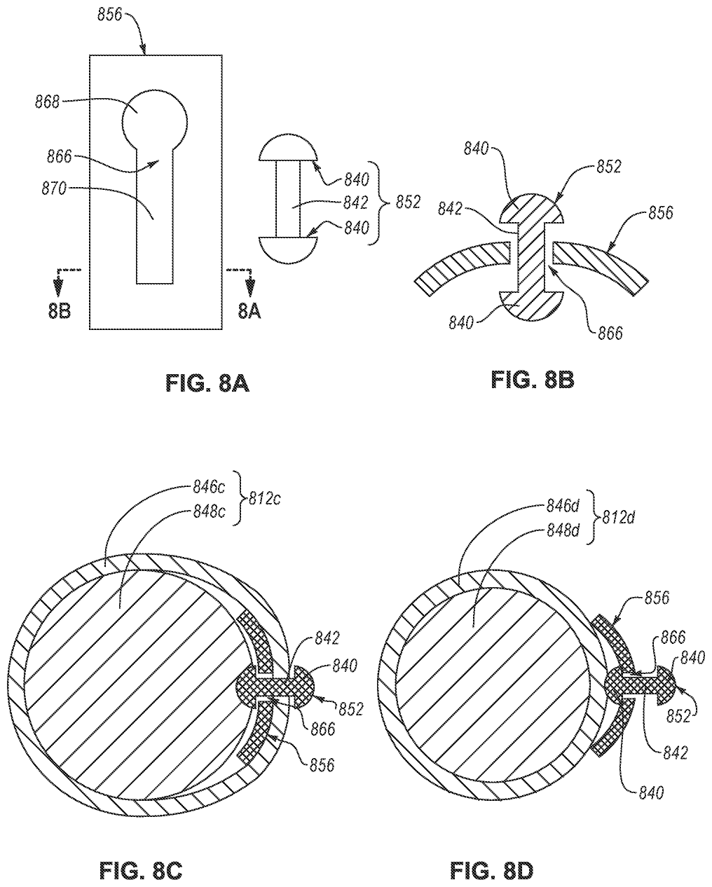

Another example of a second coupling that is formed from two or more distinct components is illustrated in FIGS. 8A and 8B. FIG. 8A is a top plan view of a second coupling 852 that includes the fastener 814 and the spine 856 separated, according to an embodiment. FIG. 8B is a cross-sectional schematic of the second coupling 852 fully assembled, wherein the cross-sectional is taken along plane 8B-8B of the spine 856 illustrated in FIG. 8A. Referring to FIG. 8A, the fastener 814 includes two heads 840 and a rod 842 extending between the two heads 840. The spine 856 defines a slit 866 formed therein. The slit 866 includes a first portion 868 and a second portion 870. The first portion 868 is configured to allow the head(s) 840 to pass therethrough. The second portion 870 is configured to receive the rod 842 and to prevent the heads 840 from passing therethrough. In an embodiment, as illustrated, the slit 866 is completely enclosed by the spine 866. In such an embodiment, the first portion 868 exhibits a lateral dimension that is sufficiently large to receive the head(s) 840 of the fastener 814 and the second portion 870 exhibits a lateral dimension that is sufficiently large to receive the rod 842 and sufficiently small to no receive the head(s) 840. In other words, the lateral dimension of the slit 866 changes. The changing lateral dimension of the slit 866 allows one of the heads 840 to be inserted through the first portion 868 such heads 840 are positioned on different side of the spine 856. The fastener 814 may then be slide along the length of the slit 866 such that the rod 842 moves from the first portion 868 to the second portion 870. FIG. 8B illustrates the configuration of the second coupling 852 when the rod 842 is positioned in the second portion 870. As shown in FIG. 8B, the heads 840 may not pass through the slit 866 when the rod 842 is positioned in the second portion 870 thereby securing the fastener 814 to the spine 856.

It is noted that the fastener 814 may be detached from the spine 856 by sliding the fastener 814 along the slit 866 such that the rod 842 moves from the second portion 870 to the first portion 868. When the rod 842 is in the first portion 868, the heads 840 may pass through the slit 866 thereby allowing the fastener 814 to be detached from the spine 856. Being able to detach the fastener 814 from the spine 856 may allow for replacement of damaged parts and allows a porous material (not shown) attached to the spine 856 to be detached (e.g., replaced after being used) from the shell without also detaching the fastener 814 from the shell. In an embodiment, not shown, the slit 866 is not completely enclosed by the spine 856. In such an embodiment, the slit 866 may extend from an edge of the spine 856.

The spine 856 allows the second coupling 852 illustrated in FIGS. 8A and 8B to be attached to a porous material in a variety of ways. For example, FIGS. 8C and 8D is cross-sectional schematics of different methods of attaching the spine 856 to a porous material, according to different embodiments. It is noted that the cross-sectional schematics of FIGS. 8C and 8D are taken along a plane that is traverse to the longitudinal axis of the porous material. Except as otherwise disclosed herein, the porous materials illustrated in FIGS. 8C and 8D are the same or substantially similar to any of the porous materials disclosed herein. The porous materials and second couplings 852 illustrated in FIGS. 8C and 8D may be used in any of the fluid collection assemblies disclosed herein.

Referring to FIG. 8C, the spine 856 may be disposed within the porous material 812c, such as between the fluid permeable membrane 846c and the fluid permeable support 848c of the porous material 812c. Disposing the spine 856 within the porous material 812c may allow the spine 856 to be secured to the porous material 812c without using an adhesive, weld, sewn joint, etc. Not securing the spine 856 to porous material 812c with an adhesive, weld, sewn joint, etc. also allows the spine 856 to slide within the porous material 812c. Sliding the spine 856 within the porous material 812c may move the rod 842 from a first portion 868 to a second portion 870 (the first and second portions 868, 880 are illustrated in FIG. 8A) or vice versa. Disposing the spine 856 within the porous material 812c may require forming a recess in the porous material 812c for the fastener 814 to be positioned through. Referring to FIG. 8D, the spine 856 may be attached to an outer surface of the porous material 812d. For example, as illustrated, the spine 856 may be attached to an outer surface of the fluid permeable membrane 846. The spine 856 may be attached to the outer surface of the porous material 812d using an adhesive, welding, sewing, or any other suitable technique.

Similar to the fasteners 114 illustrated in FIG. 1B, the fasteners of the second couplings illustrated in FIGS. 3-8D may block the perforation formed in the shell that is configured to allow air to flow into the chamber. As such, the second couplings illustrated in FIGS. 3-8 may also define one or more perforations such that air may flow through the shell. For example, FIG. 9 is a cross-sectional schematic of a second coupling 952 that defines one or more passageways 944 extending therethrough, according to an embodiment. Except as otherwise disclosed herein, the second coupling 952 may be the same or substantially similar to any of the second couplings disclosed herein and may be used in any of the fluid collection assemblies disclosed herein. The second coupling 952 includes one or more fasteners 914 and a spine 956. At least one of the fastener 914 define a passageway 944 extending at least partially therethrough as previously discussed herein. For example, the passageway 944 may extend from the head 940 of the fastener 914 since the head 940 may be exposed to an atmosphere outside of the chamber defined by the shell. The passageway 944 may extend from the head 940 and through at least a portion of the rod 942 to a portion of the second coupling 952 that is in fluid communication with the chamber. In an embodiment, as illustrated, the passageway 944 may extend through a portion of the spine 956. Extending the passageway 944 through the spine 956 may allow the passageway 944 to follow a generally linear path which may facilitate forming the passageway 944 through the fastener 914. For example, the linear path of the passageway 944 may allow the passageway 944 to be formed by drilling or may allow the passageway 944 to be formed using injection molding.

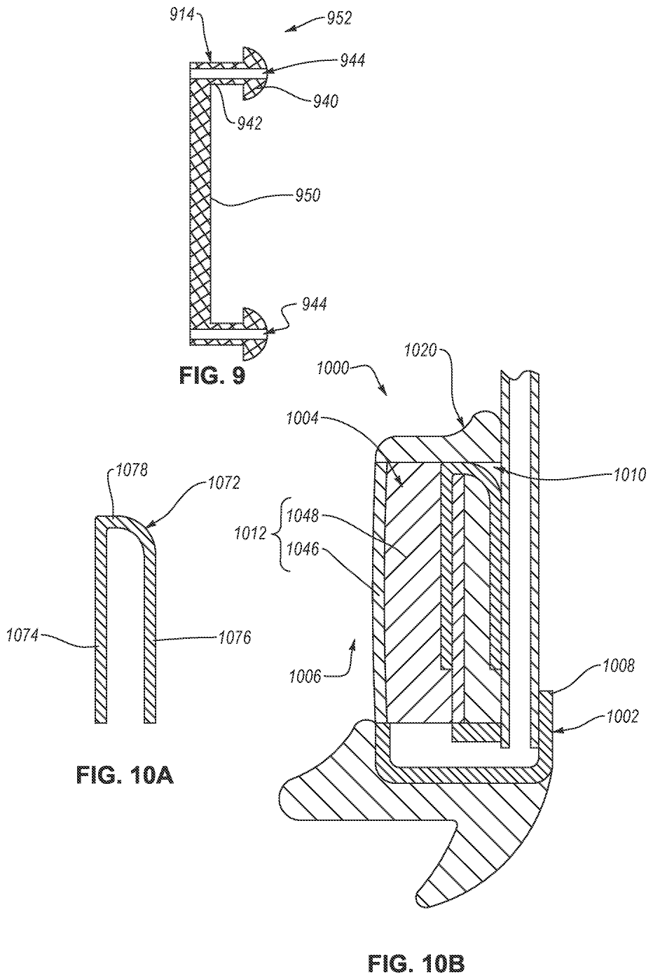

The second couplings disclosed herein may include an element other than or in addition to a fastener. For example, FIG. 10A is a side view of a second coupling that is a clip 1072, according to an embodiment. The clip 1072 may include a first arm 1074, a second arm 1076, and a bend 1078 extending between the first arm 1074 and the second arm 1076. The clip 1072 is configured to secure the porous material 1012 to the fluid impermeable barrier 1002.

FIG. 10B is a cross-sectional schematic of a fluid collection assembly 1000 that includes the clip 1072, according to an embodiment. Except as otherwise disclosed herein, the fluid collection assembly 1000 is the same or substantially similar to any of the fluid collection assemblies disclosed herein. For example, the fluid collection assembly may include a fluid impermeable barrier 1002 that defines a chamber 1004, an opening 1006, and a fluid outlet 1008. The fluid impermeable barrier 1002 also includes a shell 1020 and a first coupling. In the illustrated embodiment, the first coupling includes an aperture 1010 formed in the shell 1020. The fluid collection assembly 1000 also includes at least one porous material 1012.

As previously discussed, the clip 1072 is configured to secure the porous material 1012 to the fluid impermeable barrier 1002. The first arm 1074 is configured to be attached to the porous material 1012. For example, the first arm 1074 may be at least partially disposed in the porous material 1012 (e.g., between the fluid permeable membrane 1046 and the fluid permeable support 1048) or attached to an outer surface of the porous material 1012 (e.g., using an adhesive, welding, sewing, etc.). The second arm 1076 is configured to be attached to the fluid impermeable barrier 1002. For example, the second arm 1076 may be at least partially disposed through the aperture 1010 formed in the shell 1020. Thus, the clip 1072 secures the porous material 1012 to the fluid impermeable barrier 1002.

Referring back to FIG. 10A, in an embodiment, the clip 1072 may exhibit a first shape when no external forces are applied to the clip 1072. When the clip 1072 exhibits the first shape, the first and second arms 1074, 1076 may be spaced from each other by a first distance. The clip 1072 may be configured to exhibit a second shape. The second shape may be formed by a force that pulls the first and second arms 1074, 1076 apart. When the clip 1072 exhibits the second shape, the first and second arms 1074, 1076 may be spaced from each other by a second distance that is greater than the first distance. The clip 1072 may be formed from a material that is sufficiently elastic that the clip 1072 only elastically deforms when the clip 1072 switches from the first shape to the second shape. As such, the clip 1072 may desire to return the first shape. It is noted that the clip 1072 may include one or more additional shapes, such as a third shape when the distance between the first and second arms 1074, 1076 is greater than the first distance and greater than or less than the second distance.

As shown in FIG. 10A, the clip 1072 may exhibit the first shape when the clip 1072 is not securing the porous material 1012 to the fluid impermeable barrier 1002. In an embodiment, as illustrated in FIG. 10B, the clip 1072 may exhibit the second shape when the clip 1072 is securing the porous material 1012 to the fluid impermeable barrier 1002. In other words, the components of the fluid collection assembly 1000 between the first arm 1074 and the second arm 1076 is greater than the first distance. As such, the first and second arms 1074, 1076 may apply a compressive force to the porous material 1012 and/or the fluid impermeable barrier 1002. The compressive force may minimize movement between the porous material 1012 and the fluid impermeable barrier 1002 during use. In an embodiment, the clip 1072 may exhibit the second shape during the act of attaching the porous material 1012 to the fluid impermeable barrier 1002. In such an embodiment, the clip 1072 may need to be deformed while attaching the porous material 1012 to the fluid impermeable barrier 1002. After attaching the porous material 1012 to the fluid impermeable barrier 1002, the clip 1072 may revert back to the first shape or form a third shape where the distance between the first and second arms 1074, 1076 is greater than the first distance and less than the second distance.

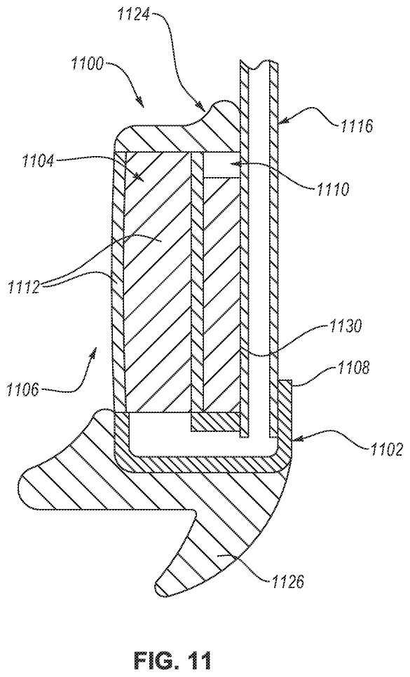

As previously discussed, the fluid collection assemblies disclosed herein may include a fluid impermeable barrier defining at least one aperture instead of or in addition to the aperture forming the first coupling. For example, FIG. 11 is a cross-sectional view of a fluid collection assembly 1100 including a fluid impermeable barrier 1102 defining a vent 1110 (i.e., aperture) at or near a proximal end region 1124 thereof, according to an embodiment. Except as otherwise disclosed herein, the fluid collection assembly 1100 is the same or substantially similar to any of the fluid collection assemblies disclosed herein. For example, the fluid impermeable barrier 1102 may define a chamber 1104, at least one opening 1106, and a fluid outlet 1108 and the fluid collection assembly 1100 may include at least one porous material 1112 disposed in the chamber 1104.

The vent 1110 is positioned at or near the proximal end region 1124 of the fluid impermeable barrier 1102. Such a position of the vent 1110 prevents or at least inhibits leakage of bodily fluids from the chamber 1104. For example, as previously discussed, bodily fluids enter the chamber 1104 through the opening 1106 and flow through the porous material 1112 towards the fluid outlet 1108 and the conduit 1116 due to the vacuum source provided from the conduit 1116 to the chamber 1104 and due to gravity. As such, bodily fluids are significantly more likely to be present at and/or pool in portions of the chamber 1104 that are closer to the distal end region 1126 than the proximal end region 1124. Conventional fluid collection assemblies may include a vent at or near the distal end regions thereof and the bodily fluids may leak through such vents due to the presence and pooling of the bodily fluids at or near the distal end regions of the conventional fluid collection assemblies. However, the decreased quantities of bodily fluids adjacent to the vent 1110 compared to the vents of the conventional fluid collection assemblies causes the bodily fluids to be significantly less likely to leak through the vent 1110 than the vents of the conventional fluid collection assemblies.

Since bodily fluids are less likely to leak through the vent 1110, the vent 1110 may exhibit a minimum cross-sectional area that may be larger than vents of at least some conventional fluid collection assemblies. For example, the vent 1110 may exhibit a minimum cross-sectional area that is about 7.5 mm² to about 15 cm², about 10 mm² to about 20 cm², about 15 mm² to about 25 cm², about 20 mm² to about 30 cm², about 25 mm² to about 35 cm², about 30 mm² to about 40 cm², about 35 mm² to about 45 cm², about 40 mm² to about 50 cm², about 45 mm² to about 55 cm², about 50 mm² to about 60 cm², about 55 mm² to about 65 cm², about 60 mm² to about 70 cm², about 65 mm² to about 75 cm², about 70 mm² to about 80 cm², or about 75 mm² to about 85 cm². Generally, increasing the minimum cross-sectional area of the vent 1110 increases the quantity of air that may flow through the vent 1110 and through the chamber 1104 which, in turn, increases the quantity of bodily fluids that flows towards the fluid outlet 1118. Further, increasing the minimum cross-sectional area of the vent 1110 weakens the proximal end region 1124 of the fluid impermeable barrier 1102 which allows the fluid impermeable barrier 1102 to better conform to the shape of the vaginal region of the patient which may make the fluid collection assembly 1100 more comfortable to use. However, increasing the minimum cross-sectional area of the vent 1110 increases the likelihood that bodily fluids leak through the vent 1110.

In an embodiment, the vent 1110 includes a water-impermeable and air-permeable membrane configured to prevent or at least inhibit bodily fluids flowing therethrough. In an embodiment, the vent 1110 does not include a water-impermeable and air-permeable membrane since bodily fluids are less likely to leak through the vent 1110 due to the positioning thereof. It is noted that not including a water-impermeable and air-permeable membrane in the vent 1110 may simplify manufacturing of the fluid collection assembly 1100 by reducing the number of parts that make the fluid collection assembly 1100 and preempting the need to attach the membrane to the fluid collection assembly 1100.

The position of the vent 1110 at or near the proximal end region 1124 also facilitates air flow through the chamber 1104. For example, air pulled into the chamber 1104 through the vent 1110 will flow from the vent 1110 to the fluid outlet 1108. Thus, air from the vent 1110 may flow through substantially all of the chamber 1104. Air flow through substantially all of the chamber 1104 may promote flow of the bodily fluids towards the fluid outlet 1108 and inhibits pooling of bodily fluids in portions of the chamber 1104 at or near the proximal end region 1126. Meanwhile, conventional fluid collection assemblies that include vents at or near the proximal end regions thereof substantially only cause air flow in the small portions of the chamber between the vent and the fluid outlet. Thus, the air flow through the chamber 1104 caused by the vents of such conventional fluid collection assemblies does not promote fluid flow through the chamber 1104 as well as the vent 1110.

In an embodiment, not shown, the vent 1110 is at the proximal end region 1124 of the fluid impermeable barrier 1102. In such an embodiment, the vent 1110 may extend from the chamber 1104 to the proximal end region 1124. In an embodiment, as shown, the vent 1110 is near the proximal end region 1124. In such an embodiment, the vent 1110 may be spaced from the proximal end region 1124 (measured parallel to a longitudinal axis of the chamber 1104) by about 1 mm to about 3 mm, about 2 mm to about 4 mm, about 3 mm to about 5 mm, about 4 mm to about 6 mm, about 5 mm to about 7 mm, about 6 mm to about 8 mm, about 7 mm to about 9 mm, about 8 mm to about 10 mm, about 9 mm to about 11 mm, about 10 mm to about 12 mm, about 11 mm to about 13 mm, about 12 mm to about 14 mm, or about 13 mm to about 15 mm. When the vent 1110 is spaced from the proximal end region 1124, the vent 1110 may extend from the chamber 1104 to the back side 1130 of the fluid impermeable barrier 1102.

As previously discussed, the fluid collection assemblies may include a fluid impermeable barrier defining at least one aperture instead of or in addition to the aperture forming the first coupling and/or the aperture forming the vent. For example, FIG. 12 is an isometric view of a fluid collection assembly 1200 including a fluid impermeable barrier 1202 defining an aperture (i.e., indentation 1210) formed in at least one lateral flange 1280, according to an embodiment. Except as otherwise disclosed herein, the fluid collection assembly 1200 is the same or substantially similar to any of the fluid collection assemblies disclosed herein. For example, the fluid impermeable barrier 1202 may define at least one opening 1206 and the fluid collection assembly 1200 may include at least one porous material 1212 disposed in the fluid impermeable barrier 1202.

As previously discussed, the fluid impermeable barrier 1202 may include one or more flanges. For example, the fluid impermeable barrier 1202 may include an upper flange 1232 forming at least a portion of the proximal end region 1224 of the fluid impermeable barrier 1202, a bottom flange 1234 forming at least a portion of the distal end region 1226 of the fluid impermeable barrier 1202, and at least one lateral flange 1280 extending between the upper flange 1232 and the bottom flange 1234. In other words, the lateral flange 1280 may extend laterally from the portion of the fluid impermeable barrier 1202 defining the chamber (not shown).

It has been found that, during use of conventional fluid collection assemblies that include lateral flanges, patients are likely to grip the conventional fluid collection assemblies by the lateral flange while moving the conventional fluid collection assemblies (e.g., moving the conventional fluid collection assemblies towards or away from the vaginal region). It has also been found that the patient, especially when the patient is older, is more likely to drop the conventional fluid collection assembly when gripping the fluid collection assembly by the lateral flanges than if the patient gripped other portions of the conventional fluid collection assembly. Dropping the conventional fluid collection assembly may cause the conventional fluid collection assembly to become contaminated. Also, dropping the conventional fluid collection assembly after the conventional fluid collection assembly is used may cause bodily fluids present in the chamber to leak from the chamber.

The fluid collection assembly 1200 includes at least one indentation 1210 (i.e., aperture) formed in a portion lateral flange 1280. The indentation 1210 may extend partially through the lateral flange 1280 or completely through the lateral flange 1280. The indentation 1210 forms a location that the patient may grip while using the fluid collection assembly 1200. The indentation 1210 helps the patient grip the fluid collection assembly 1210 without dropping the fluid collection assembly 1200 by increasing the surface area of the lateral flange 1280 that contacts the fingers of the patient and by moving the patient's fingers closer to the portions of the fluid impermeable barrier 1200 that defines the chamber. It is noted that the indentation 1210 may facilitate an individual other than the patient (e.g., a medical practitioner) grip the fluid collection assembly 1200 without dropping the fluid collection assembly 1200.

The indentation 1210 may facilitate manipulating of the fluid collection assembly 1200. For example, as previously discussed, the fluid collection assembly 1200 may be bent such that the fluid collection assembly 1200 conforms to the shape of the vaginal region of the patient. It has been found that the indentation 1210 facilitates bending the fluid collection assembly 1200 by weakening a portion of the flanges. In other words, the indentation 1210 forms a portion of the fluid collection assembly 1200 that preferentially bends. As such, the indentation 1210 may be formed on a portion of the fluid impermeable barrier 1202 that is likely to bend when conforming the fluid collection assembly 1200 to the vaginal region. For example, the indentation 1210 may be located closer to the proximal end region 1224 of the fluid impermeable barrier 1202 than the distal end region 1226. In such an example, the indentation 1210 may be at a location of the fluid impermeable barrier 1202 that is more likely to be bend when conforming the fluid collection assembly 1200 to the vaginal region.

In an embodiment, the fluid impermeable barrier 1200 defines only single indentation 1210. In an embodiment, the fluid impermeable barrier 1200 defines two indentations 1210 on opposing side of the fluid collection assembly 1200. In such an embodiment, the two indentations 1210 facilitate gripping the fluid collection assembly 1200 by pinching the fluid collection assembly 1200 between two fingers (e.g., between the thumb and the index finger). In an embodiment, the fluid impermeable barrier 1202 defines a single indentation 1210 on one side and a plurality of indentations on the opposing side. In such an embodiment, the single indentation 1210 may be gripped with the thumb and the plurality of indentations 1210 may be gripped by one or more other finders. In an embodiment, the fluid impermeable barrier 1202 defines a plurality of indentations 1210 on both sides of the fluid collection assembly 1200 which allows the fluid collection assembly 1200 to be gripped ambidextrously.

The indentation 1210 may include one or more features that facilitates gripping the fluid collection assembly 1200. For example, FIG. 13 is a front elevational view of a fluid collection assembly 1300, according to an embodiment. Except as disclosed herein, the fluid collection assembly 1300 is the same as the fluid collection assembly 1200 of FIG. 12. The fluid impermeable barrier 1302 includes one or more protrusions 1382 extending from portions of the fluid impermeable barrier 1302 that define the indentation 1310. The protrusions 1382 further facilitate gripping the fluid collection assembly 1300. It is noted that other portions of the lateral flange 1380 may include the protrusions 1382, even when the lateral flange 1380 does not define the indentation 1310.

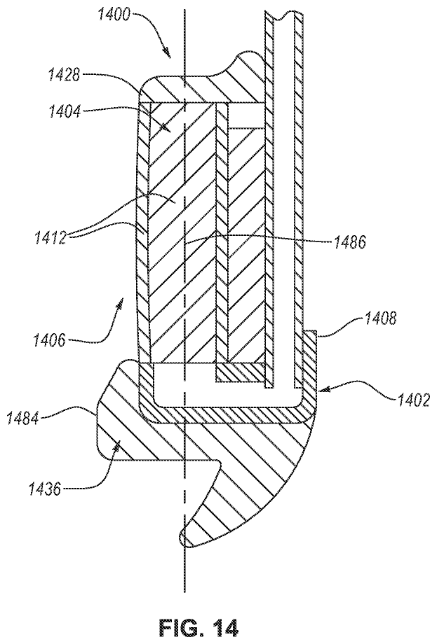

As previously discussed, the fluid collection assemblies disclosed herein may include a flattened sump instead of or in addition to any of the apertures disclosed herein. FIG. 14 is cross-sectional view of a fluid collection assembly 1400, according to an embodiment. Except as otherwise disclosed herein, the fluid collection assembly 1400 is the same as or substantially similar to any of the fluid collection assemblies disclosed herein. For example, the fluid collection assembly 1400 includes a fluid impermeable barrier 1402 defining at least a chamber 1404, at least one opening 1406, and a fluid outlet 1408. The fluid collection assembly 1400 also includes at least one porous material 1412 disposed in the chamber 1404.

The fluid impermeable barrier 1402 includes a sump 1436 at a distal end region 1426 of the fluid impermeable barrier 1402. The sump 1436 may extend outwardly from the front side 1428 of the fluid impermeable barrier 1402. Extending the sump 1436 outwardly allows the sump 1436 to interface with the vaginal opening of the patient to facilitate correct positioning of the fluid collection assembly 1400 on the patient.

The sump 1436 includes a flattened surface 1484. The flattened surface 1484 may be generally parallel (±30°) to a longitudinal axis 1486 of the chamber 1404 and is generally flat. The flattened surface 1484 is generally flat when the flattened surface 1484 exhibits at least one of an average radius of curvature that is significantly less than the surrounding portions of the fluid impermeable barrier 1402 or exhibits an average radius of curvature that is greater than about 7.5 cm (e.g., greater than about 10 cm, greater than about 15 cm, or greater than about 20 cm). The flattened surface 1484 of the sump 1436 decreases the likelihood that the sump 1436 penetrates the vaginal opening compared to other positional features. As such, the flattened surface 1484 may make using the fluid collection assembly 1400 more comfortable since some patients find having a sump or other positional feature penetrate the vaginal opening to be uncomfortable.

The sump 136 may extend outwardly from the front side 1428 and/or the porous material 112 by a distance d. The distance d may be about 1 mm to about 3 mm, about 2 mm to about 4 mm, about 3 mm to about 5 mm, about 4 mm to about 6 mm, about 5 mm to about 7 mm, about 6 mm to about 8 mm, about 7 mm to about 9 mm, about 8 mm to about 10 mm, about 9 mm to about 11 mm, about 10 mm to about 12 mm, about 11 mm to about 13 mm, about 12 mm to about 14 mm, or about 13 mm to about 15 mm.

It is noted that the apertures and flattened sump may be used with fluid collection assemblies other than the fluid collection assemblies illustrated in FIGS. 1A-2, 8C, 8D, and 10B-14. Examples of fluid collection assemblies that may include any of the apertures disclosed herein and/or the flattened sump are disclosed in U.S. patent application Ser. No. 16/433,773 filed on Jun. 6, 2019, U.S. Pat. No. 10,226,376 filed on Jun. 1, 2017, and International Application No. PCT/US21/39866 filed on Jun. 30, 2021, the disclosures of each of which are incorporated herein, in its entirety, by this reference.

FIG. 15 is a block diagram of a fluid collection system 1590 for fluid collection, according to an embodiment. The fluid collection system 1590 includes a fluid collection assembly 1500, a fluid storage container 1592, and a vacuum source 1594. The fluid collection assembly 1500 may be the same or substantially similar to any of the fluid collection assemblies disclosed herein. The fluid collection assembly 1500, the fluid storage container 1592, and the vacuum source 1594 may be fluidly coupled to each other via one or more conduits 1516. For example, fluid collection assembly 1500 may be operably coupled to one or more of the fluid storage container 1592 or the vacuum source 1594 via the conduit 1516. The bodily fluids collected in the fluid collection assembly 1500 may be removed from the fluid collection assembly 1500 via the conduits 1516 which protrudes into the fluid collection assembly 1500. For example, an inlet of the conduits 1516 may extend into the fluid collection assembly 1500, such as to a reservoir therein. The outlet of the conduits 1516 may extend into the fluid collection assembly 1500 or the vacuum source 1594. Suction force may be introduced into the chamber of the fluid collection assembly 1500 via the inlet of the conduits 1516 responsive to suction (e.g., vacuum) force applied at the outlet of the conduits 1516.

The suction force may be applied to the outlet of the conduits 1516 by the vacuum source 1594 either directly or indirectly. The suction force may be applied indirectly via the fluid storage container 1592. For example, the outlet of the conduits 1516 may be disposed within the fluid storage container 1592 and an additional conduits 1516 may extend from the fluid storage container 1592 to the vacuum source 1594. Accordingly, the vacuum source 1594 may apply suction to the fluid collection assembly 1500 via the fluid storage container 1592. The suction force may be applied directly via the vacuum source 1594. For example, the outlet of the conduits 1516 may be disposed within the vacuum source 1594. An additional conduits 1516 may extend from the vacuum source 1594 to a point outside of the fluid collection assembly 1500, such as to the fluid storage container 1592. In such examples, the vacuum source 1594 may be disposed between the fluid collection assembly 1500 and the fluid storage container 1592.

The fluid storage container 1592 is sized and shaped to retain bodily fluids therein. The fluid storage container 1592 may include a bag (e.g., drainage bag), a bottle or cup (e.g., collection jar), or any other enclosed container for storing bodily fluids such as urine. In some examples, the conduits 1516 may extend from the fluid collection assembly 1500 and attach to the fluid storage container 1592 at a first point therein. An additional conduits 1516 may attach to the fluid storage container 1592 at a second point thereon and may extend and attach to the vacuum source 1594. Accordingly, a vacuum (e.g., suction) may be drawn through fluid collection assembly 1500 via the fluid storage container 1592. Bodily fluids, such as urine, may be drained from the fluid collection assembly 1500 using the vacuum source 1594.

The vacuum source 1594 may include one or more of a manual vacuum pump, and electric vacuum pump, a diaphragm pump, a centrifugal pump, a displacement pump, a magnetically driven pump, a peristaltic pump, or any pump configured to produce a vacuum. The vacuum source 1594 may provide a vacuum or suction to remove bodily fluids from the fluid collection assembly 1500. In some examples, the vacuum source 1594 may be powered by one or more of a power cord (e.g., connected to a power socket), one or more batteries, or even manual power (e.g., a hand operated vacuum pump). In some examples, the vacuum source 1594 may be sized and shaped to fit outside of, on, or within the fluid collection assembly 1500. For example, the vacuum source 1594 may include one or more miniaturized pumps or one or more micro pumps. The vacuum sources 1594 disclosed herein may include one or more of a switch, a button, a plug, a remote, or any other device suitable to activate the vacuum source 1594.

While various aspects and embodiments have been disclosed herein, other aspects and embodiments are contemplated. The various aspects and embodiments disclosed herein are for purposes of illustration and are not intended to be limiting.

Terms of degree (e.g., “about,” “substantially,” “generally,” etc.) indicate structurally or functionally insignificant variations. In an example, when the term of degree is included with a term indicating quantity, the term of degree is interpreted to mean±20%, ±5%, or ±2% of the term indicating quantity. In an example, when the term of degree is used to modify a shape, the term of degree indicates that the shape being modified by the term of degree has the appearance of the disclosed shape. For instance, the term of degree may be used to indicate that the shape may have rounded corners instead of sharp corners, curved edges instead of straight edges, one or more protrusions extending therefrom, is oblong, is the same as the disclosed shape, etc.

1. A fluid collection assembly, comprising:
a fluid impermeable barrier including a proximal end region and a distal end region opposite the proximal end region, the fluid impermeable barrier at least defining a chamber, at least one opening, and a fluid outlet at or near the distal end region, the fluid impermeable barrier defining at least one aperture, the at least one aperture forming at least one first coupling;
at least one porous material disposed in the chamber; and
at least one second coupling attached to the at least one porous material;
wherein the at least one first coupling is configured to have the at least one second coupling extend therethrough to anchor the at least one porous material in the chamber.

2. (canceled)

3. The fluid collection assembly of claim 3, wherein the at least one first coupling is at least one perforation defined by and extending through the fluid impermeable barrier.

4. The fluid collection assembly of claim 3, wherein the at least one second coupling includes at least one fastener extending through the at least one perforation.

5. The fluid collection assembly of claim 4, wherein the at least one fastener defines at least one passageway extending therethrough.

6. The fluid collection assembly of claim 4, wherein the at least one fastener includes at least one head and a rod extending from the at least one head.

7. The fluid collection assembly of claim 4, wherein the at least one fastener includes a first fastener positioned at or near a proximal end region of the at least one porous material and a second fastener positioned at or near a distal end region of the at least one porous material, the proximal end region is opposite the distal end region.

8. The fluid collection assembly of claim 4, further comprising an elongated spine attached to or integrally formed with the at least one fastener.

9. The fluid collection assembly of claim 8, wherein the elongated spine exhibits a crinkle structure including one or more ridges and defining one or more valleys.

10. The fluid collection assembly of claim 8, wherein the at least one porous material defines one or more perforations extending therethrough, and wherein the at least one porous material is bent and the at least one fastener is positioned through the one or more perforations.

11. The fluid collection assembly of claim 8, wherein the elongated spine is directly attached to the at least one porous material.

12. The fluid collection assembly of claim 8, wherein the elongated spine is ultrasonically welded to the at least one porous material.

13. The fluid collection assembly of claim 8, wherein the elongated spine includes at least one of acrylonitrile butadiene styrene, a polycarbonate acrylonitrile butadiene styrene blend or copolymer, polyethylene, polypropylene, or polycarbonate.

14. The fluid collection assembly of claim 4, further comprising a spine defining at least one slit therein, the at least one slit including a first portion and a second portion extending from the first portion, wherein the first portion exhibits a lateral dimension that is greater than the second portion, the slit configured to receive the at least one second coupling.

15. The fluid collection assembly of claim 14, wherein the at least one slit is completely enclosed by the spine.

16. The fluid collection assembly of claim 3, wherein the second coupling includes a clip, clip including a first arm, a second arm, and a bend extending between the first arm and the second arm.

17. The fluid collection assembly of claim 16, wherein the first arm is configured to be at least partially disposed within the at least one porous material and the second arm is configured to be positioned through the at least one perforation.

18. The fluid collection assembly of claim 1, wherein the fluid impermeable barrier defines at least one vent at or near the proximal end region.

19. The fluid collection assembly of claim 18, wherein the at least one vent exhibits a cross-sectional area that is about 7.5 mm² to about 85 mm².

20. The fluid collection assembly of claim 1, wherein the fluid impermeable barrier defines at least one indentation formed in the at least one lateral flange.

21. The fluid collection assembly of claim 20, wherein the fluid impermeable barrier includes one or more protrusions extend laterally outwardly from the portions of the fluid impermeable barrier defining the at least one indentation.

22. The fluid collection assembly of claim 1, wherein the fluid impermeable barrier defines a sump at or near the distal end region, the sump extending outwardly from the front side of the fluid impermeable barrier, the at least one sump including the at least one flattened surface.

23. The fluid collection assembly of claim 1, wherein the at least one porous material undefines a bore configured to receive a conduit.

24. The fluid collection assembly of claim 1, wherein the at least one porous material is free from an adhesive therein and thereon.

25. A fluid collection system, comprising:
a fluid collection assembly including:
a fluid impermeable barrier including a proximal end region and a distal end region opposite the proximal end region, the fluid impermeable barrier at least defining a chamber, at least one opening, and a fluid outlet at or near the distal end region, the fluid impermeable barrier defining at least one aperture, the at least one aperture forming at least one first coupling;
at least one porous material disposed in the chamber; and
at least one second coupling attached to the at least one porous material;
wherein the at least one first coupling is configured to have the at least one second coupling extend therethrough to anchor the at least one porous material in the chamber;
a fluid storage container; and
a vacuum source;
wherein the chamber of the fluid collection assembly, the fluid storage container, and the vacuum source are in fluid communication with each other, so that when one or more bodily fluids are present in the chamber, a suction provided from the vacuum source to the chamber of the fluid collection assembly removes the one or more bodily fluids from the chamber and deposits the bodily fluids in the fluid storage container.

26. A fluid collection assembly, comprising:
a fluid impermeable barrier including a proximal end region and a distal end region opposite the proximal end region, the fluid impermeable barrier at least defining a chamber, at least one opening, and a fluid outlet at or near the distal end region, the fluid impermeable barrier including a sump at the distal end region thereof, the sump extending outwardly from a front side of the fluid impermeable barrier, the front side of the fluid impermeable barrier defining at least a portion of the opening, the sump including at least one flattened surface; and
at least one porous material disposed in the chamber.

27. A fluid collection assembly, comprising:
a fluid impermeable barrier including a proximal end region and a distal end region opposite the proximal end region, the fluid impermeable barrier at least defining a chamber, at least one opening, and a fluid outlet at or near the distal end region, the fluid impermeable barrier including at least one lateral flange, the at least one lateral flange defining at least one indentation; and
at least one porous material disposed in the chamber.

28. The fluid collection assembly of claim 27, wherein the fluid impermeable barrier includes one or more protrusions extend laterally outwardly from the portions of the fluid impermeable barrier defining the at least one indentation.
